(19) United States
(12) Patent Application Publication
(10) Pub. No.: US 2011/0077620 A1
(43) Pub. Date: Mar. 31, 2011

(54) Guide Catheters

(76) Inventor: Nicholas C. deBeer, Montara, CA (US)

(21) Appl. No.: 12/570,128

(22) Filed: Sep. 30, 2009

(51) Int. Cl. A61M 25/00

(52) U.S. Cl. 604/524

(57) ABSTRACT

High performance guide catheters are described as suitable for neurovascular access. They are constructed using a machined core, in which the core includes transition features from a proximal to distal-most section of the device.

### FIELD OF THE INVENTION

The subject matter described herein relates generally to catheters for the delivery of diagnostic or therapeutic agents and devices to internal target sites that can be accessed through the circulatory system.

### BACKGROUND OF THE INVENTION

In pursuing endovascular treatment of a disease state, the target site which one wishes to access using one or more catheters is often set within soft tissue, such as in the brain or liver, and can only reached by a tortuous route through small vessels or ducts. In such cases, access lumen size often tapers to less than about 3 mm.

As observed by many and addressed early-on in such patents as U.S. Pat. Nos. 4,739,768 and 5,308,342 for Target Therapeutics, Inc., the difficulty in accessing such regions stems from the requirement that catheter must be quite flexible in order to follow the tortuous path into the tissue, while stiff enough to allow the distal end of the catheter to be manipulated from an external access site that may be as much as a meter or more from the treatment site.

The Target-assigned patents primarily describe microcatheter construction including multi-flex zones. Multi-flex zones have now been implemented in guide catheter products by Pnumbra, Inc. in their NEURON line of intracranial access products. The guide catheters are used to support and/or allow easier passage for a microcatheter ultimately accessing the target site to provide therapy. Accordingly, these guide catheters range in from 5 to 6 French in size with a lumen size between 0.040 and 0.070 inches.

While the Pnumbra devices have been well-received, their typical braid/coil-reinforced laminated construction limits performance possibilities. Technology offering potential for higher performance (be it in simple compression, bending and/or torsional loading) is described in U.S. Pat. No. 6,428,489 assigned to Precision Vascular Systems, Inc. The slit/cut hypotube technology described therein has been adapted to catheter construction as described in commonly-assigned US Patent publication No. 2008/0077119. In this implementation, however, the tip of the catheter is not reinforced by the slotted hypotube, but purely polymeric or optionally supported by a separate coil or braided structure.

In contrast, the present invention utilizes a reinforcing hypotube cut with patterns as described below to provide for continuous support from the proximal end of the catheter to the distal end (i.e., well past the termination point in the '119 publication devices and thereby contrary to the publication's teaching). The performance advantages so-provided will offer clinicians a valuable tool for endovascular therapy.

### SUMMARY OF THE INVENTION

Catheters according to the present invention include a processed hollow structural core, typically captured between an inner PTFE liner and PEBAX jacket (which may carry a hydrophilic coating) running substantially the entire length of the system.

The core may comprise any of stainless steel, Nitinol or another metal or alloy hypotube. It may alternatively comprise a high strength polymeric member (e.g., Polyimide or PEEK). To offer relevant performance characteristics, at least the distal portion of the structural tube is machined (e.g., by laser, EDM, and/or chemical etching processes) to increase flexibility. A proximal hub is also typically included in the overall catheter assembly.

The distal-most section of the structural core is cut into a spiral form. The spiral represents a continuous helical beam. Proximal to this section, the beam is connected by bridges at intervals of between about 210 and about 270 degrees.

This transition section is met by a more proximal section in which the beam may or may not spiral along the length of the catheter (i.e., the beam or beam sections may be helically disposed or flat/radially aligned). In any case, these beam sections are connected by bridges at intervals of less than the previous section, typically less than 180 degrees and more typically less than about 120 degrees, even less than 90 degrees apart. More proximal yet, the bridge interval may decrease and/or the beam width increase to provide a stiffer section for increased proximal shaft pushability.

The inner (working) lumen in the device is typically at least about 0.040 inches, though it is often larger (e.g., about 0.070 inches). The axial length of the various sections may vary and optimization for a given application is with in the level of skill in the art.

In the transition section, it is important that the connecting bridge elements do not align. Rather, they are staggered along with radial frequency that provides for little to substantially no preference in directional orientation thereby delivering consistent performance in vascular tortuosity.

More than simply offering a one-piece construction of the device shown in FIG. 11 of the '119 publication, the present invention adds features. Not to be bound by a particular theory, but it is thought that the exceptional performance across and including the transition zone in the subject invention derives from progressive alteration to the structure base structure.

In other words, from the coil section to the transition section, only bridges are added. The beam configuration does not change or does not substantially change. Then from the transition to the proximal section, bridge frequency is increased as well as beam width. So, variation in the architecture of the structural core transitions first in one domain, then in two domains to achieve the desirable performance. In this sense, the nature of invention is one of taking the simplest functional form for the distal-most section (i.e., the helical coil) and compounding differences for functional benefit in transition to the proximal portion of the catheter body.

Other systems, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE DRAWINGS

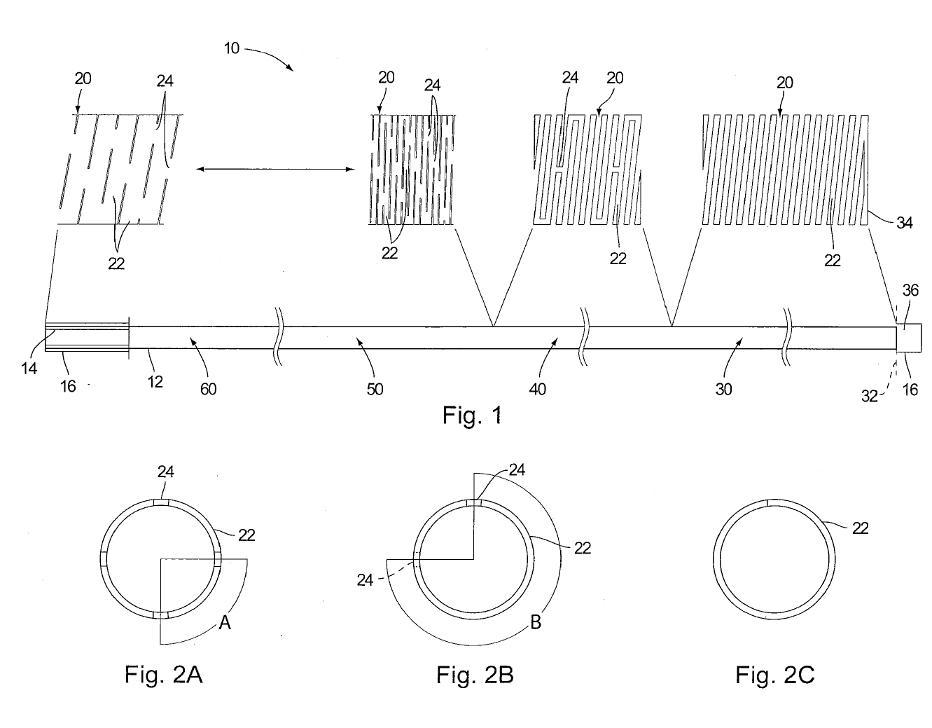

The details of the inventive subject matter set forth herein, both as to its structure and operation, may be appreciated in part by study of the accompanying figures, in which like reference numerals refer to like parts. The components in the figures are not necessarily to scale, with emphasis instead being placed upon illustrating the principles of the subject invention or inventions. Moreover, all illustrations are intended to convey concepts, where relative sizes, shapes and other detailed attributes may be illustrated schematically rather than literally or precisely. That said, FIG. 1 is a partial side-section view, including detail illustrations of the subject catheter; and FIGS. 2A-2C are partial end-section view of a core member of the subject catheter.

### DETAILED DESCRIPTION

FIG. 1 illustrates details of the subject catheter. Catheter body 10 comprises a structural core 12, an inner liner 14 and an outer jacket 16. The core comprises a processed hypotube cut by conventional techniques to form slits 20 defining adjacent beams 22 and bridges 24 between the beams.

A distal extent 32 (indicated by broken line) of distal section 30 of the core typically terminates in a circumferential beam 34 at the end of the catheter indicated in broken line 34 where the liner 14 and jacket 16 are fused together to form an atraumatic tip 36.

A distinguishing feature of the present catheter is the manner in which the structural core extends substantially to the end of the device. Namely, it typically extends to within about 0.050 to about 0.1 inches of the end of the catheter. Only an optional “soft tip” structure (e.g., polymeric tip 36 or the like) extends beyond the structural core.

To allow requisite flexibility to provide for tracking in tortuous anatomy, even with such a construction, section 30 of the structural core is cut into a spiral form. The spiral represents a continuous helical beam 22 as likewise illustrated in FIG. 2C.

Proximal to this section, in a transition section 40 the beam 22 is connected by bridges 24. The interval between the bridges is about 210 and about 270 degrees (e.g., as illustrated at 270 degree intervals “B” in FIG. 2B). Transition section 40 is met by a more proximal section 50 in which the bridges 24 are more closely spaced. Typically they are spaced at about 120 degrees or less (e.g., as illustrated at 90 degree intervals “A” in FIG. 2A). As further illustrated in FIG. 1, in a each of section 50 and a more proximal section 60, the bridge 24 interval may decrease and/or the beam width 22 increase to provide a stiffer section(s) for increased shaft pushability.

In one exemplary embodiment, section 30 is about 8 to about 18 cm long and section 40 is about 1 to about 2 cm long. Together, sections 50 and 60 are about 90 to about 100 cm long with a notable transition between configurations approximately as shown in FIG. 1 (indicated by the double-headed arrow). In this exemplary embodiment, the structural core comprises stainless steel hypotube (304/316 alloy) with a 0.00275″ wall thickness). However, while an embodiment of the invention is described with reference to one or more numerical values, these values are intended as examples only and in no way should be construed as limiting the subject matter recited in any claim, absent express recitation of a numerical value in that claim.

And while the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, to the extent multiple equivalent species are described herein, recitation of an individual species in the recited claims should not be interpreted as a donation of the subject matter of the unrecited species to the public. Also, to the extent equivalent species are not recited herein, this should not be interpreted as an express or implied admission that said unrecited species are not in fact equivalents, or that said unrecited species would not be obvious to one of ordinary skill in the art after reading this disclosure.

1. A catheter comprising:
a machined structural core, an inner polymeric liner and an outer polymeric jacket,
a distal-most section of the core consisting of a continuous helical beam,
an adjacent transition section of the core comprising a helical beam section connected by bridges at intervals of between about 210 and about 270 degrees, and
a proximal section of the core adjacent to the transition section comprising beam sections connected by bridge sections at intervals of between about 120 and 90 degrees.

2. The catheter of claim 1, wherein the helical beam is at least substantially unchanged between the distal most section and the transition section.

3. The catheter of claim 2, wherein the beam sections in the proximal section are stiffer than those in the transition and distal-most sections.

4. The catheter of claim 1, wherein the proximal beam sections are arranged in a helical pattern.

5. The catheter of claim 1, wherein the liner and jacket are fused distal to the distal-most core section.

6. The catheter of claim 5, wherein the fused section is about 0.1 inches or less in length.
